SEQUENCE LISTING (9 sequences)

SEQ ID NO: 1 - DNA, 40 bp, Homo sapiens
caagcagttttcagttcctcttcggaggaaatcatgtctt

SEQ ID NO: 2 - DNA, 2055 bp, Homo sapiens
atggccatggactcgtcgcttcaggcccgcctgtttcccggtctcgctatcaagatccaacgcagtaatggtttaattcacagtgccaatgtaaggactgtgaacttggagaaatcctgtgtttcagtggaatgggcagaaggaggtgccacaaagggcaaagagattgattttgatgatgtggctgcaataaacccagaactcttacagcttcttcccttacatccgaaggacaatctgcccttgcaggaaaatgtaacaatccagaaacaaaaacggagatccgtcaactccaaaattcctgctccaaaagaaagtcttcgaagccgctccactcgcatgtccactgtctcagagcttcgcatcacggctcaggagaatgacatggaggtggagctgcctgcagctgcaaactcccgcaagcagttttcagttcctcttcggaggaaatcatgtcttgtgaaggaagtggaaaaaatgaagaacaagcgagaagagaagaaggcccagaactctgaaatgagaatgaagagagctcaggagtatgacagtagttttccaaactgggaatttgcccgaatgattaaagaatttcgggctactttggaatgtcatccacttactatgactgatcctatcgaagagcacagaatatgtgtctgtgttaggaaacgcccactgaataagcaagaattggccaagaaagaaattgatgtgatttccattcctagcaagtgtctcctcttggtacatgaacccaagttgaaagtggacttaacaaagtatctggagaaccaagcattctgctttgactttgcatttgatgaaacagcttcgaatgaagttgtctacaggttcacagcaaggccactggtacagacaatctttgaaggtggaaaagcaacttgttttgcatatggccagacaggaagtggcaagacacatactatgggcggagacctctctgggaaagcccagaatgcatccaaagggatctatgccatggcctcccgggacgtcttcctcctgaagaatcaaccctgctaccggaagttgggcctggaagtctatgtgacattcttcgagatctacaatgggaagctgtttgacctgctcaacaagaaggccaagctgcgcgtgctggaggacggcaagcaacaggtgcaagtggtggggctgcaggagcatctggttaactctgctgatgatgtcatcaagatgctcgacatgggcagcgcctgcagaacctctgggcagacatttgccaactccaattcctcccgctcccacgcgtgcttccaaattattcttcgagctaaagggagaatgcatggcaagttctctttggtagatctggcagggaatgagcgaggcgcagacacttccagtgctgaccggcagacccgcatggagggcgcagaaatcaacaagagtctcttagccctgaaggagtgcatcagggccctgggacagaacaaggctcacaccccgttccgtgagagcaagctgacacaggtgctgagggactccttcattggggagaactctaggacttgcatgattgccacgatctcaccaggcataagctcctgtgaatatactttaaacaccctgagatatgcagacagggtcaaggagctgagcccccacagtgggcccagtggagagcagttgattcaaatggaaacagaagagatggaagcctgctctaacggggcgctgattccaggcaatttatccaaggaagaggaggaactgtcttcccagatgtccagctttaacgaagccatgactcagatcagggagctggaggagaaggctatggaagagctcaaggagatcatacagcaaggaccagactggcttgagctctctgagatgaccgagcagccagactatgacctggagacctttgtgaacaaagcggaatctgctctggcccagcaagccaagcatttctcagccctgcgagatgtcatcaaggccttacgcctggccatgcagctggaagagcaggctagcagacaaataagcagcaagaaacggccccagtga

SEQ ID NO: 3 - protein, 684 aa, Homo sapiens [sequence omitted]

SEQ ID NO: 4 - DNA, 20 bp, Homo sapiens
tcagttcctcttcggaggaa

SEQ ID NO: 5 - protein, 10 aa, Homo sapiens
QFSVPLRRKS

SEQ ID NO: 6 - DNA, 28 bp, Homo sapiens
ttcttggtattgcgcgtttctcttcctt

SEQ ID NO: 7 - DNA, 28 bp, Homo sapiens
agttctgggccttcttctcttctcgctt

SEQ ID NO: 8 - DNA, 27 bp, Homo sapiens
atggccatggactcgtcgcttcaggcc

SEQ ID NO: 9 - DNA, 27 bp, Homo sapiens
tcactggggccgtttcttgctgcttat

(19) United States
(12) Patent Application Publication
(10) Pub. No.: US 2006/0177828 A1
(43) Pub. Date: Aug. 10, 2006

(54) Alternatively spliced isoform of mitotic centromere-associated kinesin (MCAK)

(76) Inventors: Christopher D. Armour, Kirkland, WA (US); John C. Castle, Seattle, WA (US); Philip W. Garrett-Engele, Seattle, WA (US); Zhengyan Kan, Bellevue, WA (US); Patrick M. Loerch, Boston, MA (US); Nicholas F. Tsinoremas, Sammamish, WA (US)

Correspondence Address: R. Douglas Bradley;Merck & Co., Inc., Patent Department RY60-30, P.O. Box 2000, Rahway, NJ, 07065-0907, US

(21) Appl. No.: 10/942,521

(22) Filed: Sep. 16, 2004

Related U.S. Application Data
(60) Provisional application No. 60/504,553, filed on Sep. 18, 2003.

(51) Int. Cl. C12Q 1/68; C07H 21/04; C12P 21/06; C12N 9/00

(52) U.S. Cl. 435/6; 435/69.1; 435/183; 435/320.1; 435/325; 536/23.2

(57) ABSTRACT

The present invention features a nucleic acid and a polypeptide encoding a novel splice variant isoform of mitotic centromere-associated kinesin (MCAK). The polynucleotide sequence of MCAKsv1 is provided by SEQ ID NO 2. The amino acid sequence for MCAKsv1 is provided by SEQ ID NO 3. The present invention also provides methods for using MCAKsv1 polynucleotide and protein to screen for compounds that bind to MCAKsv1 and to screen for compounds that bind to other kinesin isoform polypeptides but not to MCAKsv1.

This application claims priority to U.S. Provisional Patent Application Ser. No. 60/504,553 filed on Sep. 18, 2003, which is incorporated by reference herein in its entirety.

### BACKGROUND OF THE INVENTION

The references cited herein are not admitted to be prior art to the claimed invention.

Kinesins are a functionally diverse superfamily of ATP-dependent microtubule based motor proteins that have been implicated in the movement of chromosomes (Yen et al., 1992, Nature 359: 536-539), the movement of membrane-bound organelles (Hall and Hedgecock, 1991, Cell 65: 837-847), the regulation of microtubule and spindle pole dynamics (Nislow et al., 1992, Nature 359: 543-547; Sawin and Mitchison, 1991, J. Cell Biol. 112: 941-954), the assembly and maintenance of flagella (Morris and Scholey, 1997, J. Cell Biol. 138: 1009-1022), and the depolymerization of microtubules (Walczak et al., 1996, Cell 84: 37-47; Desai et al., 1999, Cell 96: 69-78; Hunter and Wordeman, 2000, J. Cell Sci. 113: 4379-4389; Kinoshita et al., 2001, Science 294: 1340-1343; Maney et al., 2001, J. Biol. Chem. 276: 34753-34758). Most kinesin heavy chains contain three major domains arranged in a modular fashion: the motor domain, neck, and stalk. Structural variants of the kinesin heavy chain are classified based on the position of the motor domain. The motor of N-type variants is located at the amino terminus while C-type variants have the motor domain at the carboxy terminus of the molecule. The motor domain in I-type variants is in an internal position.

The Kin I family (I-type kinesins) includes mitotic centromere-associated kinesin (MCAK; also called KNSL6 and KIF2C) from human (Kim et al., 1997, Biochim. Biophys. Acta 1359: 181-186) and its homologues: MCAK from hamster (Wordeman and Mitchison, 1995, J. Cell Biol. 128: 95-104), mKIF2 from mouse (Noda et al., 1995, J. Cell Biol. 129: 157-167; Santama et al., 1998, EMBO J. 17: 5855-5867), XKCM1 from Xenopus (Walczak et al., 1996), and FKIF from fish (Bost-Usinger et al., 1997, Exp. Eye Res. 64: 781-794). MCAK is classified as a kinesin-related protein based on the high degree of homology of its motor domain with other members of the kinesin superfamily (Wordeman and Mitchison, 1995; Vale and Fletterick, 1997, Ann. Rev. Cell Dev. Biol. 13: 745-777).

While most kinesins move cargo by translocating along the surface of microtubules, MCAK and its homologues depolymerize microtubules from either end (Walczak et al., 1996; Desai et al., 1999; Hunter and Wordeman, 2000; Kinoshita et al., 2001; Maney et al., 2001). Addition of purified, recombinant MCAK to microtubules that have been stabilized by taxol or the slowly hydrolyzed GTP analog GMP-CPP causes almost complete loss of polymer in the presence of ATP after 10-30 minutes (Desai et al., 1999; Maney et al., 2001; Niederstrasser et al., 2002, J. Mol. Biol. 316: 817-828; Moores et al., 2002, Mol. Cell 9: 903-909). Furthermore, when microtubules are incubated with Kin I kinesins in the presence of the nonhydrolyzable ATP analog AMP-PNP, electron micrographs reveal curled protofilament structures, intermediate structures in the depolymerization pathway, at their ends (Desai et al., 1999; Moores et al., 2002; Mandelkow et al., 1991, J. Cell Biol. 114: 977-991; Muller-Reichert et al., 1998, Proc. Natl. Acad. Sci. USA 95: 3661-3666). Consistent with the microtubule depolymerization activity demonstrated in vitro, depletion or inhibition of Xenopus XKCM1 increases the size of the mitotic spindle in egg extracts (Walczak et al., 1996; Tournebize et al., 2000, Nat. Cell Biol. 2: 13-19).

MCAK has also been implicated in the proper alignment of chromosomes at the metaphase plate. A MCAK protein construct lacking the motor domain is able to bind to centromeres and displace endogenous MCAK in CHO cells (Maney et al., 1998, J Cell Biol. 142: 787-801). Displacement of endogenous MCAK from the centromeres by the motorless MCAK results in a lagging chromosome phenotype during anaphase chromosome movement (Maney et al., 1998). Antisense RNA-induced depletion of MCAK in CHO cells results in the same lagging chromosome phenotype, indicating that functional MCAK is required for proper chromosome segregation (Maney et al., 1998). Likewise, displacement of endogenous XKCM1 by an N-terminal fragment of XKCM1 caused a misalignment of chromosomes on the metaphase plate without affecting global spindle structure (Walczak et al., 2002, Curr. Biol. 12: 1885-1889). Because chromosome movement during anaphase is associated with the shortening of microtubules, the lagging chromosome phenotype observed in MCAK depleted cells may be related to the role of MCAK in microtubule depolymerization (Hunter et. al., 2003, Mol. Cell 11: 445-457). MCAK localization to the centromere could also help establish and maintain proper kinetochore microtubule attachments during chromosome congression (the process by which chromosomes move toward the metaphase plate) by enhancing depolymerization of microtubules that are not properly attached to the kinetochore (Walczak et al., 2002).

Analysis of MCAK deletion constructs has helped define which domains of the MCAK protein are required for centromere association and microtubule depolymerization. The minimal XKCM1 domain that results in effective targeting to the centromere is contained in the first 149 amino acids of the XKCM1 protein (Walczak et al., 2002). These findings are consistent with studies showing that the N-terminal domain of MCAK is essential for centromere targeting in CHO cells (Wordeman et al., 1999, Cell Biol. Int. 23: 275-286; Maney et al., 1998). Thus, the N-terminus of MCAK is required for association of MCAK with the centromere.

The motor domain of hamster MCAK is necessary but not sufficient for microtubule depolymerization (Maney et al., 2001). An additional 31 amino acids N-terminal to the motor domain (in the neck region of the protein) are required to restore efficient microtubule depolymerization (Maney et al., 2001). Although the neck region itself does not possess depolymerizing activity (Maney et al., 1998), alanine substitutions of highly conserved positively charged residues in the MCAK neck domain also significantly reduce microtubule depolymerization activity in vitro (Ovechkina et al., 2002, J. Cell Biol. 159: 557-562). Thus, the neck may interact electrostatically with tubulin to permit diffusional translocation of MCAK along the microtubule (Ovechkina et al., 2002).

Human MCAK is expressed as a 2.9 kilo base (kb) message in dividing cells (Kim et al., 1997). Human MCAK is expressed at high levels in thymus and testis; at low levels in small intestine, colon (mucosal lining), and placenta; and very weakly in spleen and ovary (Kim et al., 1997). There is no detectable human MCAK specific mRNA in prostrate, peripheral blood leukocytes, heart, brain, lung, liver, skeletal muscle, kidney, and pancreas (Kim et al., 1997). This data suggests that human MCAK mRNA is transcriptionally regulated and is expressed primarily in proliferating tissues.

MCAK protein localization varies throughout the cell cycle. In interphase, hamster MCAK is located both in the nucleus and in the cytoplasm (Wordeman and Mitchison, 1995; Maney et al., 1998). At prophase, hamster MCAK becomes concentrated throughout the centromere region and between the kinetochore plates where it persists until telophase (Wordeman and Mitchison, 1995; Maney et al., 1998). A similar localization pattern has been observed in Xenopus. XKCM1 protein, the Xenopus MCAK homologue, is also found in the cytoplasm and associated with the centromeres and spindle poles (Walczak et al., 1996). Consistent with the localization of MCAK to centromeres, human MCAK physically associates with CENP-H, a centromere associated protein, in vitro (Sugata et al., 2000, Hum. Mol. Genet. 9: 2919-2926).

A number of compounds known to modulate kinesin activity have been disclosed. For example, U.S. Pat. No. 6,489,134 and U.S. Pat. No. 6,207,403 disclose compounds derived from the marine sponge Adocia that are effective modulators of kinesin motors. Furthermore, other compounds, such as phenothiazine and triphenylmethane, that inhibit kinesins, including KSP, have previously been described (U.S. Pat. No. 6,613,540; U.S. Pat. No. 6,440,686; WO 02/057244; WO 02/056880; WO 03/050122). Methods for screening for compounds that modulate kinesin activity based on monitoring the fluorescence of tryptophan residues or monitoring the effect of the compound on bioactivity (such as cellular proliferation, viablility, motility, morphology, ATP hydrolysis, or bipolar spindle formation) have also been disclosed (U.S. Pat. No. 6,613,540; U.S. Pat. No. 6,440,686; U.S. Pat. No. 6,410,254). Methods for using anti-sense oligonucleotides to modify the expression of MCAK and other kinesins have been described previously (WO 03/030832; Maney et al., 1998, J. Cell Biol. 142: 787-801).

Given the unique role of human MCAK in regulating microtubule dynamics and chromosome segregation, it is likely that that MCAK is an important contributor to cell cycle progression (Zhai et al., 1996, J. Cell Biol. 135: 201-214) and tumorgenesis (Laksmi et al., 1993, Anti-cancer Res. 13: 299-303). Many human cancers have been linked to chromosomal instability that leads to an abnormal number of chromosomes (aneuploidy) (Lengauer et al., 1997, Nature 386: 623-627). Significantly, mRNA expression levels of human MCAK were found to be upregulated in 9 of 9 colon cancer specimens, with the level of mRNA expression ranging from 5 to 44 times the level detected in normal colon tissue, indicating that MCAK could be a valuable therapeutic target for colon cancer (Scanlan et al., 2002, Cancer Res. 62: 4041-4047).

### BRIEF DESCRIPTION OF THE FIGURES

FIG. 1A illustrates the exon structure of MCAK mRNA corresponding to the known long reference form of MCAK mRNA (labeled NM_{—}006845.1) and the exon structure corresponding to the inventive short form splice variant (labeled MCAKsv1). FIG. 1B depicts the nucleotide sequence of an exon junction region (SEQ ID NO 1) resulting from splicing of exon 5 to exon 7 in MCAKsv1 mRNA. In FIG. 1B, in sequence shown as SEQ ID NO 1, the nucleotides shown in italics represent the 20 nucleotides at the 3′ end of exon 5 and the nucleotides shown in underline represent the 20 nucleotides at the 5′ end of exon 7.

### SUMMARY OF THE INVENTION

Microarray experiments and RT-PCR have been used to identify and confirm the presence of a novel splice variant of human MCAK mRNA. More specifically, the present invention features polynucleotides encoding a new protein isoform of MCAK. A polynucleotide sequence encoding MCAKsv1 is provided by SEQ ID NO 2. An amino acid sequence for MCAKsv1 is provided by SEQ ID NO 3.

Thus, a first aspect of the present invention describes a purified MCAKsv1 encoding nucleic acid. The MCAKsv1 encoding nucleic acid comprises SEQ ID NO 2 or the complement thereof. Reference to the presence of one region does not indicate that another region is not present. For example, in different embodiments the inventive nucleic acid can comprise, consist, or consist essentially of an encoding nucleic acid sequence of SEQ ID NO 2.

Another aspect of the present invention describes a purified MCAKsv1 polypeptide that can comprise, consist or consist essentially of the amino acid sequence of SEQ ID NO 3.

Another aspect of the present invention describes expression vectors. In one embodiment of the invention, the inventive expression vector comprises a nucleotide sequence encoding a polypeptide comprising, consisting, or consisting essentially of SEQ ID NO 3, wherein the nucleotide sequence is transcriptionally coupled to an exogenous promoter.

Alternatively, the nucleotide sequence comprises, consists, or consists essentially of SEQ ID NO 2, and is transcriptionally coupled to an exogenous promoter. Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 2 or a nucleotide sequence encoding a polypeptide comprising, consisting, or consisting essentially of an amino acid sequence of SEQ ID NO 3, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing MCAKsv1 polypeptide comprising SEQ ID NO 3. The method involves the step of growing a recombinant cell containing an inventive expression vector under conditions wherein the polypeptide is expressed from the expression vector.

Another aspect of the present invention features a purified antibody preparation comprising an antibody that binds selectively to MCAKsv1 as compared to one or more MCAK isoform polypeptides that are not MCAKsv1.

Another aspect of the present invention provides a method of screening for a compound that binds to MCAKsv1 or fragments thereof. In one embodiment, the method comprises the steps of: (a) expressing a polypeptide comprising the amino acid sequence of SEQ ID NO 3 or a fragment thereof from recombinant nucleic acid; (b) providing to said polypeptide a labeled MCAK ligand that binds to said polypeptide and a test preparation comprising one or more test compounds; (c) and measuring the effect of said test preparation on binding of said test preparation to said polypeptide comprising SEQ ID NO 3.

In another embodiment of the method, a compound is identified that binds selectively to MCAKsv1 polypeptide as compared to one or more kinesin isoform polypeptides that are not MCAKsv1. This method comprises the steps of: providing a MCAKsv1 polypeptide comprising SEQ ID NO 3; providing a kinesin isoform polypeptide that is not MCAKsv1; contacting said MCAKsv1 polypeptide and said kinesin isoform polypeptide that is not MCAKsv1 with a test preparation comprising one or more test compounds; and determining the binding of said test preparation to said MCAKsv1 polypeptide and to said kinesin isoform polypeptide that is not MCAKsv1, wherein a test preparation that binds to said MCAKsv1 polypeptide but does not bind to said kinesin isoform polypeptide that is not MCAKsv1 contains a compound that selectively binds said MCAKsv1 polypeptide.

In another embodiment of the invention, a method is provided for screening for a compound able to bind to or interact with a MCAKsv1 protein or a fragment thereof comprising the steps of: expressing a MCAKsv1 polypeptide comprising SEQ ID NO 3 or a fragment thereof from a recombinant nucleic acid; providing to said polypeptide a labeled MCAK ligand that binds to said polypeptide and a test preparation comprising one or more compounds; and measuring the effect of said test preparation on binding of said labeled MCAK ligand to said polypeptide, wherein a test preparation that alters the binding of said labeled MCAK ligand to said polypeptide contains a compound that binds to or interacts with said polypeptide.

Another aspect of the present invention provides a method of screening for a compound that binds to one or more kinesin isoform polypeptides that are not MCAKsv1. This method comprises the steps of: providing a MCAKsv1 polypeptide comprising SEQ ID NO 3; providing a kinesin isoform polypeptide that is not MCAKsv1; contacting said MCAKsv1 polypeptide and said kinesin isoform polypeptide that is not MCAKsv1 with a test preparation comprising one or more test compounds; and determining the binding of said test preparation to said MCAKsv1 polypeptide and to said kinesin isoform polypeptide that is not MCAKsv1, wherein a test preparation that binds to said kinesin isoform polypeptide that is not MCAKsv1 but not to said MCAKsv1 polypeptide contains a compound that selectively binds said kinesin isoform polypeptide.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “MCAK” refers to a human mitotic centromere-associated kinesin (NP 006836). In contrast, reference to an MCAK isoform includes NP_{—}006836 and other polypeptide isoform variants of MCAK.

As used herein, “MCAKsv1” refers to a splice variant isoform of human MCAK protein, wherein the splice variant has the amino acid sequence set forth in SEQ ID NO 3.

As used herein, “MCAK” refers to polynucleotides encoding MCAK.

As used herein, “MCAKsv1” refers to polynucleotides that are identical to MCAK encoding polynucleotides, except that the sequence represented by exon 6 of the MCAK messenger RNA is not present in MCAKsv1.

As used herein, “MCAKsv1” refers to polynucleotides encoding MCAKsv1 having an amino acid sequence set forth in SEQ ID NO 3.

As used herein, a “kinesin isoform” is any isoform of any kinesin from any organism, including but not limited to human chromokinesin, Kin2, Kif1A, KSP, CENP-E, MCAK, HSET, Kif15, Eg5, MKLPI, BimC, Kid and ATSV kinesins.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human MCAKsv1 that is an alternatively spliced isoform of MCAK, and to the amino acid sequences encoding this protein. SEQ ID NO 2 is a polynucleotide sequence representing an exemplary open reading frame that encodes the MCAKsv1 protein. SEQ ID NO 3 shows the polypeptide sequence of MCAKsv1.

MCAKsv1 polynucleotide sequence encoding MCAKsv1 protein, as exemplified and enabled herein includes a number of specific, substantial and credible utilities. For example, MCAKsv1 encoding nucleic acid was identified in a mRNA sample obtained from a human source (see Example 1). Such a nucleic acid can be used as a hybridization probe to distinguish between cells that produce MCAKsv1 transcript from human or non-human cells (including bacteria) that do not produce such a transcript. Similarly, antibodies specific for MCAKsv1 can be used to distinguish between cells that express MCAKsv1 from human or non-human cells (including bacteria) that do not express MCAKsv1.

MCAK is an important drug target for the management of cell cycle progression and tumorgenesis (Zhai et al., 1996, J. Cell Biol. 135: 201-214; Laksmi et al., 1993, Anti-cancer Res. 13: 299-303; Scanlan et al., 2002, Cancer Res. 62: 4041-4047). Given the potential importance of MCAK activity to the therapeutic management of a wide array of diseases, it is of value to identify MCAK isoforms and identify MCAK-ligand compounds that are isoform specific, as well as compounds that are effective ligands for two or more different kinesin isoforms. In particular, it may be important to identify compounds that are effective inhibitors of a specific kinesin isoform activity, yet do not bind to or interact with a plurality of different kinesin isoforms. Compounds that bind to or interact with multiple kinesin isoforms may require higher drug doses to saturate multiple MCAK-isoform binding sites and thereby result in a greater likelihood of secondary non-therapeutic side effects. Furthermore, biological effects could also be caused by the interaction of a drug with the MCAKsv1 isoform specifically. For the foregoing reasons, MCAKsv1 protein represents a useful compound binding target and has utility in the identification of new MCAK- and kinesin-ligands exhibiting a preferred specificity profile and having greater efficacy for their intended use.

In some embodiments, MCAKsv1 activity is modulated by a ligand compound to achieve one or more of the following: prevent or reduce the risk of occurrence, or recurrence of tumorgenesis, cancer, and disorders resulting from defects in cell cycle progression. Compounds that treat cancers are particularly important because of the cause-and-effect relationship between cancers and mortality (National Cancer Institute's Cancer Mortality Rates, http://www3.cancer.gov/atlasplus/charts.html, last visited Sep. 8, 2003).

Compounds modulating MCAKsv1 include agonists, antagonists, and allosteric modulators. While not wishing to be limited to any particular theory of therapeutic efficacy, generally, but not always, MCAKsv1 compounds will be used to modulate the activity of MCAK isoforms, thereby affecting cell cycle regulation, microtubule depolymerization, and chromosome segregation. Because many human cancers have been linked to chromosomal instability that leads to an abnormal number of chromosomes (aneuploidy) (Lengauer et al., 1997, Nature 386: 623-627), modulation of MCAK isoforms could be a valuable therapeutic target for cancer. Therefore, agents that modulate MCAK activity may be used to achieve a therapeutic benefit for any disease or condition due to, or exacerbated by, abnormal levels of MCAK protein or its activity.

MCAKsv1 activity can also be affected by modulating the cellular abundance of transcripts encoding MCAKsv1. Compounds modulating the abundance of transcripts encoding MCAKsv1 include a cloned polynucleotide encoding MCAKsv1 that can express MCAKsv1 in vivo, antisense nucleic acids targeted to MCAKsv1 transcripts, and enzymatic nucleic acids, such as ribozymes and RNAi, targeted to MCAKsv1 transcripts.

In some embodiments, MCAKsv1 activity is modulated to achieve a therapeutic effect upon diseases in which regulation of MCAK is desirable. For example, disorders resulting from defective cell cycle progression may be treated by modulating MCAKsv1 activity. In other embodiments, cancer and tumorgenesis may be treated by modulating MCAKsv1 activity.

MCAKsv1 Nucleic Acids

MCAKsv1 nucleic acids contain regions that encode for polypeptides comprising, consisting, or consisting essentially of SEQ ID NO 3. The MCAKsv1 nucleic acid has a variety of uses, such as use as a hybridization probe or PCR primer to identify the presence of MCAKsv1 nucleic acids; use as a hybridization probe or PCR primer to identify nucleic acids encoding for proteins related to MCAKsv1; and/or use for recombinant expression of MCAKsv1 polypeptides. In particular, MCAKsv1 polynucleotides do not have the polynucleotide region that consists of exon 6 of the MCAK gene.

Regions in MCAKsv1 nucleic acid that do not encode for MCAKsv1, or are not found in SEQ ID NO 2, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding MCAKsv1 related protein from different sources. Obtaining nucleic acids encoding MCAKsv1 related protein from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

MCAKsv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

MCAKsv1 Probes

Probes for MCAKsv1 contain a region that can specifically hybridize to MCAKsv1 target nucleic acids under appropriate hybridization conditions and can distinguish MCAKsv1 nucleic acids from non-target nucleic acids, in particular MCAK polynucleotides containing exon 6. Probes for MCAKsv1 can also contain nucleic acid regions that are not complementary to MCAKsv1 nucleic acids.

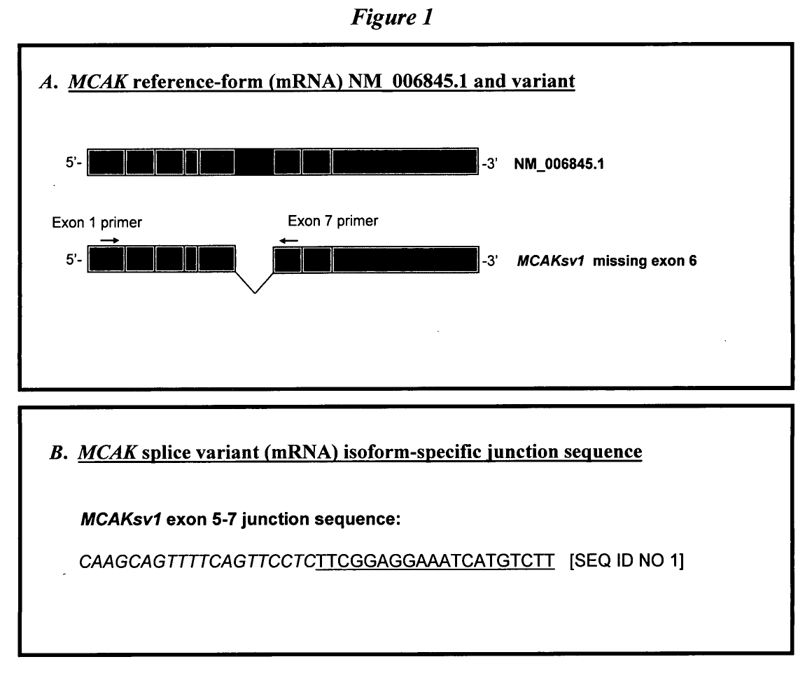

In embodiments where, for example, MCAKsv1 polynucleotide probes are used in hybridization assays to specifically detect the presence of MCAKsv1 polynucleotide in samples, the MCAKsv1 polynucleotide comprises at least 20 nucleotides of the MCAKsv1 sequence that correspond to the respective novel exon junction polynucleotide region. In particular, for detection of MCAKsv1, the probe comprises at least 20 nucleotides of the MCAKsv1 sequence that corresponds to an exon junction polynucleotide created by the alternative splicing of exon 5 to exon 7 of the primary transcript of the MCAK gene (see FIGS. 1A and 1B). For example, the polynucleotide sequence: 5′ TCAGTTCCTCTTCGGAGGAA 3′ [SEQ ID NO 4] represents one embodiment of such an inventive MCAKsv1 polynucleotide wherein a first 10 nucleotide region is complementary and hybridizable to the 3′ end of exon 5 of the MCAK gene and a second 10 nucleotide region is complementary and hybridizable to the 5′ end of exon 7 of the MCAK gene (see FIG. 1B).

In some embodiments, the first 20 nucleotides of a MCAKsv1 probe comprise a first continuous region of 5 to 15 nucleotides that is complementary and hybridizable to the 3′ end of exon 5 and a second continuous region of 5 to 15 nucleotides that is complementary and hybridizable to the 5′ end of exon 7.

In other embodiments, the MCAKsv1 polynucleotide comprises at least 40, 60, 80 or 100 nucleotides of the MCAKsv1 sequence that corresponds to a junction polynucleotide region created by the alternative splicing of exon 5 to exon 7 in the case of MCAKsv1. In embodiments involving MCAKsv1, the MCAKsv1 polynucleotide is selected to comprise a first continuous region of at least 5 to 15 nucleotides that is complementary and hybridizable to the 3′ end of exon 5 and a second continuous region of at least 5 to 15 nucleotides that is complementary and hybridizable to the 5′ end of exon 7. As will be apparent to a person of skill in the art, a large number of different polynucleotide sequences from the region of the exon 5 to exon 7 splice junction may be selected which will, under appropriate hybridization conditions, have the capacity to detectably hybridize to MCAKsv1 polynucleotide and yet will hybridize to a much less extent or not at all to MCAK isoform polynucleotides wherein exon 5 is not spliced to exon 7.

Preferably, non-complementary nucleic acid that is present has a particular purpose such as being a reporter sequence or being a capture sequence. However, additional nucleic acid need not have a particular purpose as long as the additional nucleic acid does not prevent the MCAKsv1 nucleic acid from distinguishing between target polynucleotides, e.g., MCAKsv1 polynucleotide and non-target polynucleotide including, but not limited to MCAK polynucleotide not comprising the exon 5 to exon 7 splice junction found in MCAKsv1.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

MCAKsv1 polynucleotide, such as that comprising SEQ ID NO 2 can be used to make MCAKsv1 polypeptide. In particular, MCAKsv1 polypeptide can be expressed from recombinant nucleic acids in a suitable host or in vitro using a translation system. Recombinantly expressed MCAKsv1 polypeptide can be used, for example, in assays to screen for compounds that bind MCAKsv1. Alternatively, MCAKsv1 polypeptide can also be used to screen for compounds that bind to one or more MCAK isoforms, but do not bind to MCAKsv1.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M (TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 2 to take into account codon usage of the host. Codon usage of different organisms is well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

MCAKsv1 Polypeptide

MCAKsv1 polypeptides contain an amino acid sequence comprising, consisting or consisting essentially of SEQ ID NO 3. MCAKsv1 polypeptides have a variety of uses, such as providing a marker for the presence of MCAKsv1; use as an immunogen to produce antibodies binding to MCAKsv1; use as a target to identify compounds binding selectively to MCAKsv1; use in the screening of both known and newly identified compounds affecting known kinesin drug targets, or use in an assay to identify compounds that bind to one or more kinesin isoforms but do not bind to or interact with MCAKsv1.

In chimeric polypeptides containing one or more regions from MCAKsv1 and one or more regions not from MCAKsv1, the region(s) not from MCAKsv1 can be used, for example, to achieve a particular purpose or to produce a polypeptide that can substitute for MCAKsv1 or fragments thereof. Particular purposes that can be achieved using chimeric MCAKsv1 polypeptides include providing a marker for MCAKsv1, modulating cell cycle progression, or preventing tumorgenesis and cancer.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional MCAKsv1

Functional MCAKsv1 is a different protein isoform of MCAK. The identification of the amino acid and nucleic acid sequences of MCAKsv1 provides tools for obtaining functional proteins related to MCAKsv1 from other sources, for producing MCAKsv1 chimeric proteins, and for producing functional derivatives of SEQ ID NO 3.

MCAKsv1 polypeptides can be readily identified and obtained based on their sequence similarity to MCAKsv1 (SEQ ID NO 3). In particular, MCAKsv1 lacks the amino acids encoded by exon 6 of the MCAK gene. The deletion of exon 6 and the splicing of exon 5 to exon 7 of the MCAK hnRNA transcript do not disturb the translation reading frame. However, the exon 5 to exon 7 splice junction results in a valine to leucine amino acid change at the exon 5 to exon 7 splice junction. MCAKsv1 polypeptide is also missing the amino acids encoded by exon 6 as compared to the MCAK reference sequence (NP_{—}006836).

Both the amino acid and nucleic acid sequences of MCAKsv1 can be used to help identify and obtain MCAKsv1 polypeptide. For example, SEQ ID NO 2 can be used to produce degenerative nucleic acid probes or primers for identifying and cloning nucleic acid polynucleotides encoding for a MCAKsv1 polypeptide. In addition, polynucleotides comprising, consisting, or consisting essentially of SEQ ID NO 2 or fragments thereof, can be used under conditions of moderate stringency to identify and clone nucleic acids encoding MCAKsv1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with MCAKsv1 obtained from a particular source, derivatives can be produced. Such derivatives include polypeptides with amino acid substitutions, additions and deletions. Changes to MCAKsv1 to produce a derivative having essentially the same properties should be made in a manner not altering the tertiary structure of MCAKsv1.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide than glutamate because of its long aliphatic side chain (See, Ausubel, Current Protocols in Molecular Biology, John Wiley, 1987-1998, Supplement 33 Appendix 1C).

MCAKsv1 Antibody

Antibodies recognizing MCAKsv1 can be produced using a polypeptide containing SEQ ID NO 3 or a fragment thereof, as an immunogen. Preferably, a MCAKsv1 polypeptide used as an immunogen consists of a polypeptide of SEQ ID NO 3 or a SEQ ID NO 3 fragment having at least 10 contiguous amino acids in length corresponding to the polynucleotide region representing the junction resulting from the splicing of exon 5 to exon 7 of the MCAK gene.

In some embodiments where, for example, MCAKsv1 polypeptides are used to develop antibodies that bind specifically to MCAKsv1 and not to other isoforms of MCAK, the MCAKsv1 polypeptides comprise at least 10 amino acids of the MCAKsv1 polypeptide sequence corresponding to a junction polynucleotide region created by the alternative splicing of exon 5 to exon 7 of the primary transcript of the MCAK gene (see FIG. 1). For example, the amino acid sequence: amino terminus-QFSVPLRRKS-carboxy terminus [SEQ ID NO 5] represents one embodiment of such an inventive MCAKsv1 polypeptide wherein a first 5 amino acid region is encoded by nucleotide sequence at the 3′ end of exon 5 of the MCAK gene and a second 5 amino acid region is encoded by the nucleotide sequence directly after the novel splice junction. Preferably, at least 10 amino acids of the MCAKsv1 polypeptide comprise a first continuous region of 2 to 8 amino acids that is encoded by nucleotides at the 3′ end of exon 5 and a second continuous region of 2 to 8 amino acids that is encoded by nucleotides at the 5′ end of exon 7.

In other embodiments, MCAKsv1-specific antibodies are made using a MCAKsv1 polypeptide that comprises at least 20, 30, 40 or 50 amino acids of the MCAKsv1 sequence that corresponds to a junction polynucleotide region created by the alternative splicing of exon 5 to exon 7 of the primary transcript of the MCAK gene. In each case the MCAKsv1 polypeptides are selected to comprise a first continuous region of at least 5 to 15 amino acids that is encoded by nucleotides at the 3′ end of exon 5 and a second continuous region of 5 to 15 amino acids that is encoded by nucleotides directly after the novel splice junction.

Antibodies to MCAKsv1 have different uses, such as to identify the presence of MCAKsv1 and to isolate MCAKsv1 polypeptides. Identifying the presence of MCAKsv1 can be used, for example, to identify cells producing MCAKsv1. Such identification provides an additional source of MCAKsv1 and can be used to distinguish cells known to produce MCAKsv1 from cells that do not produce MCAKsv1. For example, antibodies to MCAKsv1 can distinguish human cells expressing MCAKsv1 from human cells not expressing MCAKsv1 or non-human cells (including bacteria) that do not express MCAKsv1. Such MCAKsv1 antibodies can also be used to determine the effectiveness of MCAKsv1 ligands, using techniques well known in the art, to detect and quantify changes in the protein levels of MCAKsv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

MCAKsv1 Binding Assay

A number of compounds known to modulate kinesin activity have been disclosed. For example, U.S. Pat. No. 6,489,134 and U.S. Pat. No. 6,207,403 disclose compounds derived from the marine sponge Adocia that are effective modulators of kinesin motors. Furthermore, other compounds, such as phenothiazine and triphenylmethane, that inhibit kinesins, including KSP, have previously been described (U.S. Pat. No. 6,613,540; U.S. Pat. No. 6,440,686; WO 02/057244; WO 02/056880; WO 03/050122). Methods for screening for compounds that modulate kinesin activity based on monitoring the fluorescence of tryptophan residues or monitoring the effect of the compound on bioactivity (such as cellular proliferation, viablility, motility, morphology, ATP hydrolysis, or bipolar spindle formation) have also been described (U.S. Pat. No. 6,613,540; U.S. Pat. No. 6,440,686; U.S. Pat. No. 6,410,254). A person skilled in the art should be able to use these methods to screen MCAKsv1 or other kinesin isoform polypeptides for compounds that bind to, and in some cases functionally alter, each respective kinesin isoform protein.

MCAKsv1 or fragments thereof can be used in binding studies to identify compounds binding to or interacting with MCAKsv1 or fragments thereof. In one embodiment, MCAKsv1, or a fragment thereof, can be used in binding studies with MCAK isoform protein, or a fragment thereof, to identify compounds that: bind to or interact with MCAKsv1 and other MCAK isoforms; bind to or interact with one or more other MCAK isoforms and not with MCAKsv1. Such binding studies can be performed using different formats including competitive and non-competitive formats. Further competition studies can be carried out using additional compounds determined to bind to MCAKsv1 or other MCAK isoforms.

The particular MCAKsv1 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using MCAKsv1 expressed from a recombinant nucleic acid. Alternatively, recombinantly expressed MCAKsv1 consists of the SEQ ID NO 3 amino acid sequence.

Binding assays can be performed using individual compounds or preparations containing different numbers of compounds. A preparation containing different numbers of compounds having the ability to bind to MCAKsv1 can be divided into smaller groups of compounds that can be tested to identify the compound(s) binding to MCAKsv1.

Binding assays can be performed using recombinantly produced MCAKsv1 present in different environments. Such environments include, for example, cell extracts and purified cell extracts containing a MCAKsv1 recombinant nucleic acid; and also include, for example, the use of a purified MCAKsv1 polypeptide produced by recombinant means which is introduced into different environments.

In one embodiment of the invention, a binding method is provided for screening for a compound able to bind selectively to MCAKsv1. The method comprises the steps: providing a MCAKsv1 polypeptide comprising SEQ ID NO 3; providing a kinesin isoform polypeptide that is not MCAKsv1; contacting the MCAKsv1 polypeptide and the kinesin isoform polypeptide that is not MCAKsv1 with a test preparation comprising one or more test compounds; and then determining the binding of the test preparation to the MCAKsv1 polypeptide and to the kinesin isoform polypeptide that is not MCAKsv1, wherein a test preparation that binds to the MCAKsv1 polypeptide, but does not bind to kinesin isoform polypeptide that is not MCAKsv1, contains one or more compounds that selectively bind to MCAKsv1.

In another embodiment of the invention, a binding method is provided for screening for a compound able to bind selectively to a kinesin isoform polypeptide that is not MCAKsv1. The method comprises the steps: providing a MCAKsv1 polypeptide comprising SEQ ID NO 3; providing a kinesin isoform polypeptide that is not MCAKsv1; contacting the MCAKsv1 polypeptide and the kinesin isoform polypeptide that is not MCAKsv1 with a test preparation comprising one or more test compounds; and then determining the binding of the test preparation to the MCAKsv1 polypeptide and the kinesin isoform polypeptide that is not MCAKsv1, wherein a test preparation that binds the kinesin isoform polypeptide that is not MCAKsv1, but does not bind MCAKsv1, contains a compound that selectively binds the kinesin isoform polypeptide that is not MCAKsv1.

The above-described selective binding assays can also be performed with a polypeptide fragment of MCAKsv1, wherein the polypeptide fragment comprises at least 10 consecutive amino acids that are coded by a nucleotide sequence that bridges the junction created by the splicing of the 3′ end of exon 5 to the 5′ end of exon 7. Similarly, the selective binding assays may also be performed using a polypeptide fragment of an MCAK isoform polypeptide that is not MCAKsv1, wherein the polypeptide fragment comprises at least 10 consecutive amino acids that are coded by: a) a nucleotide sequence that is contained within exon 6 of the MCAK gene; or b) a nucleotide sequence that bridges the junction created by the splicing of the 3′ end of exon 5 to the 5′ end of exon 6 or the splicing of the 3′ end of exon 6 to the 5′ end of exon 7 of the MCAK gene.

MCAK Functional Assays

MCAK is a member of the Kin I kinesin family, characterized by the presence of an internal motor domain. MCAK depolymerizes microtubules from either end and has been implicated in the proper alignment of chromosomes at the metaphase plate. Therefore, MCAK likely plays a critical role in chromosome segregation and maintenance of proper ploidy during mitosis. The identification of MCAKsv1 as a splice variant of MCAK provides a means for screening for compounds that bind to MCAKsv1 protein thereby altering the ability of the MCAKsv1 polypeptide to depolymerize microtubules, bind to centromeres during prophase, or physically associate with CENP-H. Assays involving a functional MCAKsv1 polypeptide can be employed for different purposes, such as selecting for compounds active at MCAKsv1; evaluating the ability of a compound to effect the depolymerization of microtubules or physical association of MCAKsv1 with centromeres and CENP-H; and mapping the activity of different MCAKsv1 regions. MCAKsv1 activity can be measured using different techniques such as: detecting a change in the intracellular conformation of MCAKsv1; detecting a change in the intracellular location of MCAKsv1; detecting the amount of binding of MCAKsv1 to the centromere or to CENP-H protein; measuring microtubule depolymerization in the presence of MCAKsv1; or by measuring ATPase activity in the presence of MCAKsv1.

Recombinantly expressed MCAKsv1 can be used to facilitate the determination of whether a compound's activity in a cell is dependent upon the presence of MCAKsv1. For example, MCAKsv1 can be expressed by an expression vector in a cell line and used in a co-culture growth assay, such as described in U.S. Pat. No. 6,518,035, to identify compounds that alter the growth of the cell expressing MCAKsv1 from the expression vector as compared to the same cell line but lacking the MCAKsv1 expression vector. Alternatively, determination of whether a compound's activity on a cell is dependent upon the presence of MCAKsv1 can also be done using gene expression profile analysis methods as described, for example, in U.S. Pat. No. 6,324,479.

Techniques for measuring microtubule binding and microtubule depolymerization are well known in the art (Wordeman and Mitchison, 1995, J Cell Biol 128: 95-105; Maney et al., 2001, J Biol. Chem 276: 34753-34758; Ovechkina et al., 2002, J Cell Biol 159: 557-562; Hunter et al., 2003, Mol. Cell 11: 445-457). Methods for monitoring the ATPase activity of protein isoforms have also been described (for example, see Hunter et al., 2003, Mol. Cell 11: 445-457). Immunological techniques for assessing MCAK association with CENP-H and centromeres have also been disclosed (Wordeman et al., 1999, Cell Biol. Int. 23: 275-286; Faulkner et al., 1998, Human Mol. Genet. 7: 671-677; Walczak et al., 2002, Curr. Biol. 12: 1885-1889). A variety of other assays has been used to investigate the properties of MCAK and therefore would also be applicable to the measurement of MCAKsv1 function.

MCAKsv1 functional assays can be performed using cells expressing MCAKsv1 at a high level. These proteins will be contacted with individual compounds or preparations containing different compounds. A preparation containing different compounds where one or more compounds affect MCAKsv1 in cells over-producing MCAKsv1 as compared to control cells containing an expression vector lacking MCAKsv1 coding sequences, can be divided into smaller groups of compounds to identify the compound(s) affecting MCAKsv1 activity.

MCAKsv1 functional assays can be performed using recombinantly produced MCAKsv1 present in different environments. Such environments include, for example, cell extracts and purified cell extracts containing MCAKsv1 expressed from recombinant nucleic acid; and the use of purified MCAKsv1 produced by recombinant means that is introduced into a different environment suitable for measuring binding, ATPase activity, or microtubule depolymerizing activity.

Modulating MCAKsv1 Expression

MCAKsv1 expression can be modulated as a means for increasing or decreasing MCAKsv1 activity. Such modulation includes inhibiting the activity of nucleic acids encoding the MCAK isoform target to reduce MCAK isoform protein or polypeptide expression, or supplying MCAK nucleic acids to increase the level of expression of the MCAK target polypeptide thereby increasing MCAK activity.

Inhibition of MCAKsv1 Activity

MCAKsv1 nucleic acid activity can be inhibited using nucleic acids recognizing MCAKsv1 nucleic acid and affecting the ability of such nucleic acid to be transcribed or translated. Inhibition of MCAKsv1 nucleic acid activity can be used, for example, in target validation studies.

A preferred target for inhibiting MCAKsv1 is mRNA stability and translation. The ability of MCAKsv1 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding regions of the gene that disrupt the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8). Methods for using anti-sense oligonucleotides to modify the expression of MCAK and other kinesins have been described previously (WO 03/030832; Maney et al., 1998, J. Cell Biol. 142: 787-801).

Increasing MCAKsv1 Expression

Nucleic acids encoding MCAKsv1 can be used, for example, to cause an increase in MCAK activity or to create a test system (e.g., a transgenic animal) for screening for compounds affecting MCAKsv1 expression. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of MCAKsv1 Using Microarrays

To identify variants of the “normal” splicing of the exon regions encoding MCAK, an exon junction microarray, comprising probes complementary to each splice junction resulting from splicing of the 21 exon coding sequences in MCAK heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least one of the exon junctions of MCAK mRNA was altered in some of the tissues examined, suggesting the presence of MCAK splice variant mRNA populations. Reverse transcription and polymerase chain reaction (RT-PCR) were then performed using oligonucleotide primers complementary to exons 1 and 7 to confirm the exon junction array results and to allow the sequence structure of the splice variants to be determined.

### Example 2

### Confirmation of MCAKsv1 Using RT-PCR

The structure of MCAK mRNA in the region corresponding to exons 1 to 7 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 1 and exon 7 of the reference exon coding sequences in MCAK (NM_{—}006845.1). Based upon the nucleotide sequence of MCAK mRNA, the MCAK exon 1 and exon 7 primer set (hereafter MCAK₁₋₇ primer set) was expected to amplify a 678 base pair amplicon representing the “reference” MCAK mRNA region. The MCAK exon 1 forward primer has the sequence: 5′TTCTTGGTATTGCGCGTTTCTCTTCCTT 3′ [SEQ ID NO 6]; and the MCAK exon 7 reverse primer has the sequence: 5′AGTTCTGGGCCTTCTTCTCTTCTCG CTT 3′ [SEQ ID NO 7].

Twenty-five ng of polyA mRNA from each tissue was subjected to a one-step reverse transcription-PCR amplification protocol using the Qiagen, Inc. (Valencia, Calif.), One-Step RT-PCR kit, using the following cycling conditions:

50° C. for 30 minutes;

95° C. for 15 minutes;

35 cycles of:
94° C. for 30 seconds;
63.5° C. for 40 seconds;
72° C. for 50 seconds; then
72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least one different RT-PCR amplicon was obtained from human mRNA samples using the MCAK₁₋₇ primer set (data not shown). Every human tissue and cell line assayed except adrenal gland exhibited the expected amplicon size of 678 base pairs for normally spliced MCAK mRNA. However, in addition to the expected MCAK amplicon of 678 base pairs, all cell lines assayed, except for adrenal gland, spleen, adrenal medulla, and brain (hippocampus), also exhibited an amplicon of about 555 base pairs. The tissues in which MCAKsv1 mRNAs were detected are marked with an “x” in Table 1.

TABLE 1

| Sample | MCAKsv1 |
|---|---|
| Heart | x |
| Kidney | x |
| Liver | x |
| Brain | x |
| Placenta | x |
| Lung | x |
| Fetal Brian | x |
| Leukemia Promyelocytic (HL-60) | x |
| Adrenal Gland | |
| Fetal Liver | x |
| Salivary Gland | x |
| Pancreas | x |
| Skeletal Muscle | x |
| Brain Cerebellum | x |
| Stomach | x |
| Trachea | x |
| Thyroid | x |
| Bone Marrow | x |
| Brain Amygdala | x |
| Brain Caudate Nucleus | x |
| Brain Corpus Callosum | x |
| Ileocecum | x |
| Lymphoma Burkitt's (Raji) | x |
| Spinal Cord | x |
| Lymph Node | x |
| Fetal Kidney | x |
| Uterus | x |
| Spleen | |
| Brain Thalamus | x |
| Fetal Lung | x |
| Testis | x |
| Melanoma (G361) | x |
| Lung Carcinoma (A549) | x |
| Adrenal Medula, normal | |
| Brain, Cerebral Cortex, normal; | x |
| Descending Colon, normal | x |
| Prostate | x |
| Duodenum, normal | x |
| Epididymus, normal | x |
| Brain, Hippocamus, normal | |
| Ileum, normal | x |
| Interventricular Septum, normal | x |
| Jejunum, normal | x |
| Rectum, normal | x |

Sequence analysis of the about 555 base pair amplicon revealed that this amplicon form results from the splicing of exon 5 of the MCAK hnRNA to exon 7; that is, exon 6 coding sequence is completely absent. Thus, the RT-PCR results confirmed the junction probe microarray data reported in Example 1, which suggested that MCAK mRNA is composed of a mixed population of molecules wherein in at least one of the MCAK mRNA splice junctions is altered.

### Example 3

### Cloning of MCAKsv1

Microarray and RT-PCR data indicate that in addition to the normal MCAK reference mRNA sequence, NM_{—}006845.1, encoding MCAK protein, NP_{—}006836, one novel splice variant form of MCAK mRNA also exists in many tissues.

Clones having a nucleotide sequence comprising the splice variant identified in Example 2 (hereafter referred to as MCAKsv1) are isolated using a 5′ “forward” MCAK primer and a 3′ “reverse” MCAK primer, to amplify and clone the entire MCAKsv1 mRNA coding sequence. The 5′ “forward” primer is designed for isolation of a full length clone corresponding to the MCAKsv1 splice variant and has the nucleotide sequence of 5′ ATGGCCATGGACTCGT CGCTTCAGGCC 3′ [SEQ ID NO 8]. The 3′ “reverse” primer is designed for isolation of a full length clone corresponding to the MCAKsv1 splice variant and has the nucleotide sequence of 5′ TCACTGGGGCCGTTTCTTGCTGCTTAT 3′ [SEQ ID NO 9].

RT-PCR

The MCAKsv1 cDNA sequence is cloned using a combination of reverse transcription (RT) and polymerase chain reaction (PCR). More specifically, about 25 ng of fetal brain polyA mRNA (BD Biosciences Clontech, Palo alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the MCAK “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted MCAKsv1, a product of about 2055 base pairs. The remainder of the 50 μl PCR reactions from fetal brain is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified MCAKsv1 RT-PCR product from fetal brain is used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative MCAKsv1 clones are identified and prepared for a PCR reaction to confirm the presence of the expected MCAKsv1 exon 5 to exon 7 splice variant structure. A 25 μl PCR reaction is performed as described above (RT-PCR section) to detect the presence of MCAKsv1, except that the reaction includes miniprep DNA from the TOPO TA/MCAKsv1 ligation as a template. About 10 μl of each 25 μl PCR reaction is run on a 1% Agarose gel and the DNA bands generated by the PCR reaction are visualized and photographed on a UV light box to determine which miniprep samples have PCR product of the size predicted for the corresponding MCAKsv1 splice variant mRNA. Clones having the MCAKsv1 structure are identified based upon amplification of an amplicon band of 2055 base pairs, whereas a normal reference MCAK clone will give rise to an amplicon band of 2178 base pairs. DNA sequence analysis of the MCAKsv1 cloned DNAs confirm a polynucleotide sequence representing the deletion of exon 6 in the case of MCAKsv1.

The polynucleotide sequence of MCAKsv1 mRNA (SEQ ID NO 2) contains an open reading frame that encodes a MCAKsv1 protein (SEQ ID NO 3) similar to the reference MCAK protein (NP_{—}006836), but lacking the amino acids encoded by a 123 base pair region corresponding to exon 6 of the full length coding sequence of the reference MCAK mRNA (NM_{—}006845.1). The deletion of the 123 base pair region does not alter the protein translation reading frame in comparison to the reference MCAK protein reading frame. The MCAKsv1 protein is therefore missing an internal 41 amino acid region as compared to the reference MCAK (NP_{—}006836). This 41 amino acid region is between the domain required for centromere binding (as defined in XKCM1; Walczak et al., 2002, Curr. Biol. 12: 1885-1889) and the domain required for microtubule depolymerization (as defined in hamster MCAK; Maney et al., 2001, J Biol. Chem 276: 34753-34758). Therefore, the MCAKsv1 splice variant protein likely has the ability to bind centromeres and depolymerize microtubules. Furthermore, the valine encoded by the MCAK reference mRNA at the exon 6 to exon 7 boundary is replaced by a leucine in the MCAKsv1 protein due to the splicing of exon 5 to exon 7.

### Example 4

### Measuring the Activity of MCAKsv1

MCAKsv1 activity is measured using either an ATPase assay that measures the release of free phosphate upon hydrolysis of ATP or a DAPI assay that measures the change in fluorescence intensity of DAPI at 465 nm upon microtubule depolymerization. To assess ATPase activity, 50 μL reactions containing 80 mM K-PIPES pH 6.9, 1 mM EGTA, 1 mM DTT, 2 mM MgCl₂, 100 μg/mL BSA, 1 mM ATP, 2.0 μM microtubules (containing 2.0 μM taxol), and 100 nM MCAKsv1 are incubated at room temperature for 50 minutes (final DMSO concentration is 5%). Reactions are quenched by the addition of 50 μL of ice cold solution A (1.8 M KCl, 50 mM EDTA). Next, 150 μL of solution B (67 μg/ml quinaldine red, 0.093% polyvinyl alcohol, 4.1 mM ammonium molybdate tetrahydrate and 383 mM sulfuric acid) is added to bind to inorganic phospate produced in the reaction. Absorbance is then measured at 540 nm.

To assess MCAKsv1 activity using the DAPI assay, 200 μl reactions containing 80 mM K-PIPES pH 6.9, 37.5 mM KCl, 1 mM EGTA, 1 mM DTT, 200 μg/mL BSA, 2 mM MgCl₂, 0.25 mM ATP, 5 μM microtubules (containing 0.5 μM taxol), and 1 μM DAPI are incubated at room temperature either with or without 50 nM MCAKsv1 enzyme. DAPI fluorescence is excited at 360 nm and monitored at 465 nm in 96 well plates. To determine the extent of microtubule depolymerization from the fluorescence measurement at 465 nm, a standard curve is generated by measuring the fluorescence at 465 nm of several solutions containing varying concentrations of microtubules and tubulin such that the microtubule concentration varies from 0.5 to 5 μM, while the total concentration of protein remains constant. The standard curve is generated in buffer conditions identical to the assay conditions. The microtubule concentration with and without MCAKsv1 enzyme is then determined and compared.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 2, or the complement thereof.

2. The purified nucleic acid of claim 1, wherein said nucleic acid comprises a region encoding SEQ ID NO 3.

3. The purified nucleic acid of claim 1, wherein said nucleotide sequence encodes a polypeptide consisting of SEQ ID NO 3.

4. A purified polypeptide comprising SEQ ID NO 3.

5. The polypeptide of claim 4, wherein said polypeptide consists of SEQ ID NO 3.

6. An expression vector comprising a nucleotide sequence encoding SEQ ID NO 3, wherein said nucleotide sequence is transcriptionally coupled to an exogenous promoter.

7. The expression vector of claim 6, wherein said nucleotide sequence encodes a polypeptide consisting of SEQ ID NO 3.

8. The expression vector of claim 6, wherein said nucleotide sequence comprises SEQ ID NO 2.

9. The expression vector of claim 6, wherein said nucleotide sequence consists of SEQ ID NO 2.

10. A method for screening for a compound able to bind to MCAKsv1 comprising the steps of:
(a) expressing a polypeptide comprising SEQ ID NO 3 from recombinant nucleic acid;
(b) providing to said polypeptide a test preparation comprising one or more test compounds; and
(c) measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 3.

15. A method of screening for compounds able to bind selectively to MCAKsv1 comprising the steps of:
(a) providing a MCAKsv1 polypeptide comprising SEQ ID NO 3;
(b) providing one or more kinesin isoform polypeptides that are not MCAKsv1;
(c) contacting said MCAKsv1 polypeptide and said kinesin isoform polypeptide that is not MCAKsv1 with a test preparation comprising one or more compounds; and
(d) determining the binding of said test preparation to said MCAKsv1 polypeptide and to said kinesin isoform polypeptide that is not MCAKsv1, wherein a test preparation that binds to said MCAKsv1 polypeptide, but does not bind to said kinesin isoform polypeptide that is not MCAKsv1, contains a compound that selectively binds said MCAKsv1 polypeptide.

16. The method of claim 15, wherein said MCAKsv1 polypeptide is obtained by expression of said polypeptide from an expression vector comprising a polynucleotide encoding SEQ ID NO 3.

17. The method of claim 16, wherein said polypeptide consists of SEQ ID NO 3.

18. A method for screening for a compound able to bind to or interact with a MCAKsv1 protein or a fragment thereof comprising the steps of:
(a) expressing a MCAKsv1 polypeptide comprising SEQ ID NO 3 or fragment thereof from a recombinant nucleic acid;
(b) providing to said polypeptide a labeled MCAK ligand that binds to said polypeptide and a test preparation comprising one or more compounds; and
(c) measuring the effect of said test preparation on binding of said labeled MCAK ligand to said polypeptide, wherein a test preparation that alters the binding of said labeled MCAK ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell

21. The method of claim 18, wherein said polypeptide is expressed from an expression vector

22. The method of claim 18, wherein said MCAKsv1 ligand is an MCAK inhibitor.

23. The method of claim 21, wherein said expression vector comprises SEQ ID NO 2 or a fragment of SEQ ID NO 2.

24. The method of claim 21, wherein said polypeptide comprises SEQ ID NO 3 or a fragment of SEQ ID NO 3.
